(19) United States
(12) Patent Application Publication
(10) Pub. No.: US 2023/0293004 A1
(43) Pub. Date: Sep. 21, 2023

(54) MIXED REALITY METHODS AND SYSTEMS FOR EFFICIENT MEASUREMENT OF EYE FUNCTION

(71) Applicant: NEVADA RESEARCH & INNOVATION CORPORATION, Reno, NV (US)

(72) Inventor: Alireza TAVAKKOLI, Reno, NV (US)

(21) Appl. No.: 18/026,424

(22) PCT Filed: Sep. 15, 2021
(86) PCT No.: PCT/US21/50452

§ 371 Date: Mar. 15, 2023

Related U.S. Application Data
(60) Provisional application No. 63/079,097, filed on Sep. 16, 2020.

(51) Int. Cl. A61B 3/00; A61B 5/00; G16H 10/60; G16H 40/63; G02B 30/22

(52) U.S. Cl. CPC A61B 3/005; A61B 3/0025; A61B 5/6803; G16H 10/60; G16H 40/63; G02B 30/22; A61B 2560/0493

(57) ABSTRACT

Methods and systems for administering modular eye tests to a patient via a head mounted display (HMD) device. In some embodiments, the HMD device receives selection of a test module associated with a specific eye test which has been downloaded from an application store, provides instructions to a patient concerning the specific eye test, receives patient input data responsive to the specific eye test instructions, and stores the patient input data in a patient database. In some embodiments, the process also includes the HMD device determining that the test module was selected by the patient, determining that the specific eye test results significantly deviate from a baseline model of the patient and then transmitting the specific eye test results to an eye doctor associated with the patient.

### CROSS REFERENCE TO RELATED APPLICATION

This application is the U.S. National Stage filing of International Patent Application No. PCT/US21/50452 filed on 15 Sep. 2021, which claims the benefit of U.S. Provisional Patent Application No. 63/079,097 filed on Sep. 16, 2020, the contents of which are hereby incorporated by reference for all purposes.

### FIELD OF THE INVENTION

The invention generally relates to providing modular and/or flexible eye tests that leverage the stereo vision and eye tracking capabilities of a head mounted display (HMD), such as a virtual reality headset, to test and/or measure visual function of a patient. More specifically, in some implementations a system including a HMD is configured to efficiently administer a plurality of modular and/or flexible eye tests which inform each other to a patient and to provide assessments concerning the eye tests, wherein the modular eye tests may include one or more tests that measure visual function such as Visual Acuity (VA), Contrast Sensitivity (CS), Color Vision (CV), Stereo Vision (SV) and Visual Fields (VF).

### BACKGROUND

The field of Ophthalmology is a branch of medicine and surgery which deals with the diagnosis and treatment of human eye disorders. A partial list of the most common diseases diagnosed and treated by Ophthalmologists includes cataract, Glaucoma, Macular degeneration, Diabetic retinopathy, Dry eyes, Strabismus (misalignment/deviation of eyes), Proptosis (bulged eyes), excessive tearing (tear duct obstruction), uveitis and eye tumors. In order to diagnose patients who may have one or more of such diseases, patients may undergo eye examinations that measure for Visual acuity, Refraction, Ocular tonometry to determine intraocular pressure, Slit lamp examination and Retina examination. Ophthalmologists thus measure or test a patient's sensitivity to light in various regions of the light-sensitive retina to measure function, as well as to quantify any disorders of the eye and the retina, the optic nerve, the optic chiasm, the visual pathways to the brain, and the brain itself. In addition, visual field testing is mandatory for glaucoma diagnosis and treatment.

Various types of apparatus are known for measuring a patient's field of vision and are used by ophthalmologists and optometrists for testing purposes. Many of these instruments are relatively complex and the eye doctor must step through various functions with the patient during an eye exam. Some of the complexity involved with using such apparatus to test the eyes can tire human patients or cause them to become inattentive to visual and/or audio cues.

Head-mounted display devices, such as Virtual Reality (VR) headsets are known, and perhaps the best known use of such VR headsets is to visually simulate a user's physical presence in virtual spaces. Such simulations typically include a three-hundred and sixty (360) degree view of the user's surrounding virtual space so that when the user turns his head he or she can view different portions of the surrounding space. Head-mounted display devices have also been used for visual field testing of patients. However, there are no eye testing head mounted display (HMD) systems capable of efficiently and comprehensively testing a patient's eyes that are flexible from the standpoint of allowing an ophthalmologist, an optometrist and/or a patient to select and/or pay for eye tests that are suitable for that particular patient. Thus, the inventors recognized that there is a need for systems and methods for providing modular and/or flexible eye tests that leverage the stereo vision and eye tracking capabilities of a head mounted display (HMD), and which eye tests are easy to select and to administer to a patient.

### BRIEF DESCRIPTION OF THE DRAWINGS

Features and advantages of some embodiments of the present disclosure, and the manner in which the same are accomplished, will become more readily apparent upon consideration of the following detailed description taken in conjunction with the accompanying drawings, which illustrate preferred and example embodiments and which are not necessarily drawn to scale, wherein:

FIG. 1 is a front view of an exemplary head-mounted display (HMD) that may be worn by a patient during eye testing in accordance with some embodiments of the disclosure;

FIG. 2 is a block diagram illustrating components of an eye examination system configured for performing eye tests in accordance with some embodiments of the disclosure;

FIGS. 3A, 3B and 3C are aerial view representations of a patient utilizing a head-mounted display (HMD) to take an eye examination in accordance with some embodiments of the disclosure;

FIG. 4 is a flowchart of an eye testing process in accordance with this disclosure;

FIG. 5 is a flowchart of a process for generating recovery parameters for a patient based on one or more eye examinations of the patient in accordance with some embodiments of the disclosure;

FIG. 6 is a block diagram of an example embodiment of the components of a head-mounted display (HMD) of a type configured for operating in a manner consistent with some embodiments of the disclosure.

### DETAILED DESCRIPTION

Reference will now be made in detail to various novel embodiments, examples of which are illustrated in the accompanying drawings. The drawings and descriptions thereof are not intended to limit the invention to any particular embodiment(s). On the contrary, the descriptions provided herein are intended to cover alternatives, modifications, and equivalents thereof. In the following description, numerous specific details are set forth in order to provide a thorough understanding of the various embodiments, but some or all of the embodiments may be practiced without some or all of the specific details. In other instances, well-known process operations have not been described in detail in order not to unnecessarily obscure novel aspects. In addition, terminology used in the Detailed Description is intended to be interpreted in its broadest reasonable manner, even though it is being used in conjunction with certain examples. The terms used in this specification generally have their ordinary meanings in the art, within the context of the disclosure, and in the specific context where each term is used.

As used herein, the term “module” refers broadly to software, hardware, or firmware (or any combination thereof) components. Modules are typically functional components that can generate useful data or other output using specified input(s). A module may or may not be self-contained. An application program (sometimes called an “application” or an “app” or “App”) may include one or more modules, or a module can include one or more application programs.

In general, and for the purposes of introducing concepts of embodiments of the present disclosure, disclosed herein are mixed reality methods and systems for efficiently measuring eye functions utilizing a head mounted display (HMD). Modular eye tests are disclosed which leverage the stereo vision and eye tracking capabilities of the HMD to test and/or measure visual function. More specifically, in some embodiments a system including a HMD is configured to efficiently administer a plurality of modular eye tests to a patient. The eye tests are flexible and may inform each other to provide test results or assessments concerning one or more visual functions such as Visual Acuity (VA), Contrast Sensitivity (CS), Color Vision (CV), Stereo Vision (SV) and Visual Fields (VF). In some embodiments, the series or plurality of eye tests encompassing an eye examination for a particular patient may be selected by an Ophthalmologist, by an Optometrist or by the patient.

In some embodiments eye examinations are performed using an HMD that is configured to present images to each eye individually, and with regard to some tests to both eyes simultaneously. In some implementations, the HMD is able to control conditions, such as brightness, during an examination and thus provide accurate and reliable test results. In addition, voice recognition technology may be used to provide instructions to the patient during an eye test, and/or to replicate the conversational exchange (or portions thereof) that would typically occur between the patient and the ophthalmologist or optometrist. The HMD may also be configured to change the visual environment experienced by the patient during testing, for example, to provide an experience involving a natural setting which may cause the patient to feel less stressful. Moreover, the HMD (or another component of the overall system, such as a computer) may be configured to identify abnormal test results in real-time and, in some cases, modify the eye test and/or eye examination accordingly. For example, one or more tests scheduled to be performed during an eye examination of a particular patient can be modified or removed and/or new or different tests can be added.

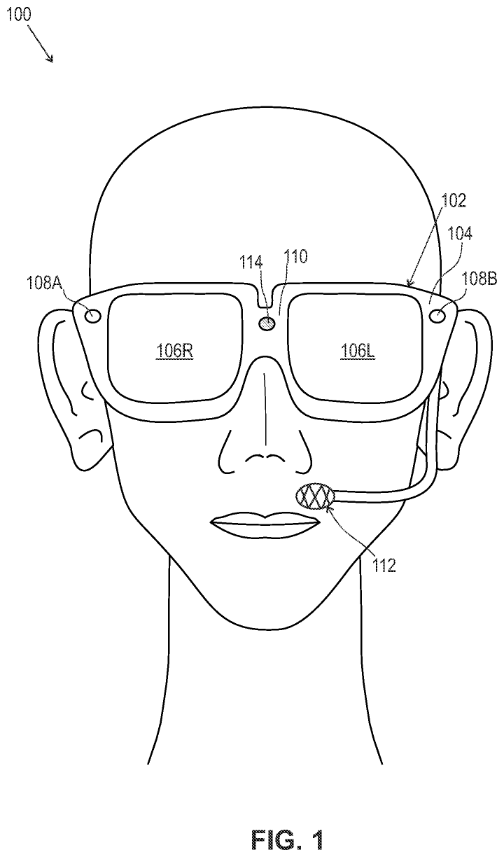

FIG. 1 is a front view of an exemplary HMD 102 that may be worn by a patient 100 in accordance with aspects of the disclosure. The HMD 102 includes a frame 104 that includes a bridge 110 configured for resting on the patient's nose. The frame 104 houses a first optical display 106L positioned in front of the left eye of the patient 100, and a second optical display 106R that is positioned in front of the right eye of the patient. The first optical display 106L and second optical display 106R are components of an image display system of the HMD 102, and both include optical display surfaces (not shown) that reflect light towards the patient's left and right eyes, respectively, and supporting electronic components (not shown). In some embodiments, the HMD 102 also includes one or more sensors 108A, 108B, a microphone 112 and a camera 114. Although the binocular HMD 102 shown in FIG. 1 resembles conventional eyeglasses, the HMD could also be in the form of goggles, or a helmet, or a visor, or a hood with first and second optical displays and the like.

The projection and presentation systems employed by HMDs can be characterized as binocular, bi-ocular, and monocular systems. Binocular systems present a separate image to each of the user's eyes, bi-ocular systems present a single image to both of the user's eyes, and monocular HMD systems present a single image to one of the user's eyes. Each of these systems or combinations thereof could be used in accordance with various types of eye tests as disclosed herein. For example, HMD 102 of FIG. 1 may utilize a binocular system and thus be capable of presenting a distinct image to each of the patient's eyes during an eye test.

In general, the HMDs described herein are configured to display simulated (e.g., computer-generated) images of a virtual environment. Thus, the HMD 102 can generate and present completely immersive “virtual reality” environments to a patient 100 during an eye examination. Convincing virtual reality images that are immersive typically require a helmet-type or goggle-type device which form-fit to a user's or patient's face and head (usually via straps, such as Velcro® straps) so that the HMD forms an enclosed area around the user's eyes. In addition, some HMDs include audio speakers such as over-ear headphones (not shown) that can be used to provide audio prompts, background music and/or atmospheric sounds and the like, while also minimizing or preventing ambient noise from being heard by the patient. Thus, an HMD in the form of goggles or a helmet prevents contamination from ambient light from entering the patient's eyes while the over ear headphones keep out ambient sound. The HMD may also include a microphone 112 to receive audio input from a patient during an eye test. It should also be understood that, in some embodiments the HMD 102 may be configured to display computer-generated (or simulated) images that are integrated into real world content perceived by the patient, which is referred to as “augmented reality” (or “AR”) and which does not require an immersive structure.

In some embodiments, a specialized HMD 102 may be used by the patient 100 that is specifically designed for performing eye examinations. In other instances, off-the-shelf HMDs may be used when configured to perform eye tests in accordance with methods disclosed herein. In particular, the various methods described below could be performed using an HMD that was designed for another purpose (for example, an HMD designed for gaming and/or entertainment purposes). For example, in some implementations in accordance with the methods disclosed herein, VR headsets manufactured by Occulus®, the HTC company, and/or Microsoft® Corporation, may be utilized in addition to traditional equipment.

Referring again to FIG. 1, as mentioned above the binocular HMD 102 includes an optical display surface 106L for the patient's left eye and an optical display surface 106R for the patient's right eye, which are configured to permit content to be presented to each of the patient's eyes individually, as well as to both the left and right eyes collectively. The optical display surfaces 106L and 106R may completely surround or wrap-around one or both eyes of the patient. For example, the frame 104 and/or bridge 110 may be designed to ensure that light (e.g., an image) presented to one eye cannot be seen by the other eye. For example, a partition (not shown) may be formed within the bridge 110 that prevents light from entering the right eye when a particular test requires testing of only the left eye, and vice-versa. Thus, the HMD 102 allows digital images to be shown to one eye, while limiting what, if anything, can be seen by the other eye of the patient depending on the requirements of a specific eye test.

The HMD 102 can also include an electronics module (not shown) for processing digital content (for example, images and/or video), for optimizing the digital content to be presented to the patient, for analyzing data collected by the one or more sensors 108A, 108B, for analyzing patient audio responses received by the microphone 112, and the like. In some implementations, one or more optical sensors (not shown) may be located within the HMD 102 and positioned to face the eyes so that such optical sensors can be utilized to detect and/or measure a patient's pupillary responses, for example, during one or more eye tests. In addition, in some embodiments the electronics module may provide at least some analysis (for example, test results) to be performed locally by the HMD 102. As will be discussed below, in some embodiments the HMD 102 may be operably connected to one or more other computing devices (such as Smart phones, tablet computers, laptop computers, server computers, and the like) that are also configured for performing some or all of such tasks. The electronics module and HMD 102 can be powered by a battery (not shown), or through a wired or wireless connection to a power source (not shown).

In some implementations, the sensors 108A, 108B coupled to the frame 104 may be operably connected to one or more of the optical display surfaces 106R, 106L and function to monitor various aspects of the patient's local environment. For example, one or both of the sensors 108A, 108B may include a temperature and/or humidity sensor for providing data associated with the comfort level in the test area for the patient and/or a light sensor which can track ambient light levels, and the like. In addition, the camera 114 may be operable to provide visual data to, for example, an eye doctor concerning the physical test room or area surrounding the patient, and/or to capture the patient's interactions with his or her environment. One or more speakers or headphones (not shown) may also be operably connected to the frame 104 and may be used to provide instructions and/or prompts to the patient during an eye examination. It should be understood that other types of sensors could also be utilized, and that the HMD 102 may also incorporate or include a hand controller (not shown) that includes motion sensing capability for capturing hand motion input from the patient during an eye test and for providing motion data which may be stored for analysis.

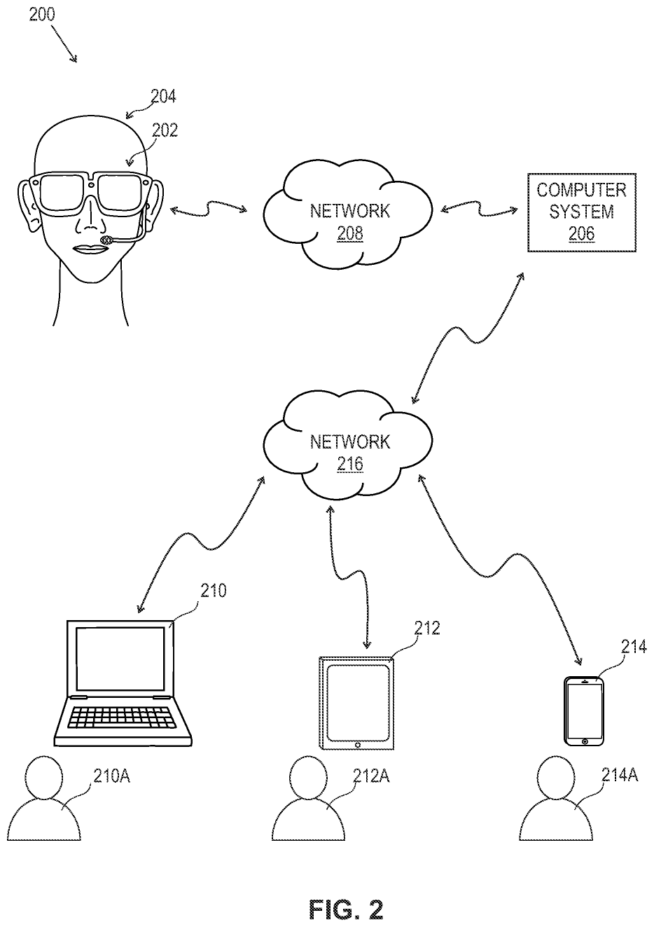

FIG. 2 is a block diagram illustrating components of an eye examination system 200 for performing eye tests in accordance with some embodiments. The HMD 202 is worn by a patient 204 and is operably connected to a computer system 206 (for example, a server computer and/or edge computing device) via a network 208, such as the Internet. The HMD 202 and/or the computer system 206 can perform some or all of the methods described herein. In particular, in some implementations the system 200 can be distributed between the HMD 204 and the computer system 206.

Referring again to FIG. 2, in some embodiments one or more additional electronic devices 210, 212, 214 that may be controlled by an ophthalmologist 210A, an optometrist 212A, and/or an optical clinician 214A and the like, are operably connected to the HMD 202 via a computer network 216 and/or to the computer system 206. The computer network 216 can be the same as, or distinct from, the network 208. In addition, the computer network 216 and/or the network 208 may be a public network (i.e., the Internet) and/or may be a private network (not shown) and/or combinations thereof. Moreover, one or more of the electronic devices 210, 212, 214 may comprise a server computer, a client computer, a personal computer (PC), a user device, a tablet computer, a laptop computer, a personal digital assistant (PDA), a cellular telephone or Smartphone, a web appliance, a wearable electronic device, a gaming device, a music player, or any electronic device capable of executing a set of instructions (sequential or otherwise) that specify actions to be taken by the electronic device in accordance with methods disclosed herein.

The eye examination system 200 permits ophthalmologists, optometrists, eye clinicians and the like to supervise the patient 204 while eye tests are being conducted. While the HMD 202, computer system 206, and the electronic devices 210, 212, 214 are depicted as wirelessly communicating with one another, in some configurations one or more of the components of the eye examination system 200 can be connected together via wires.

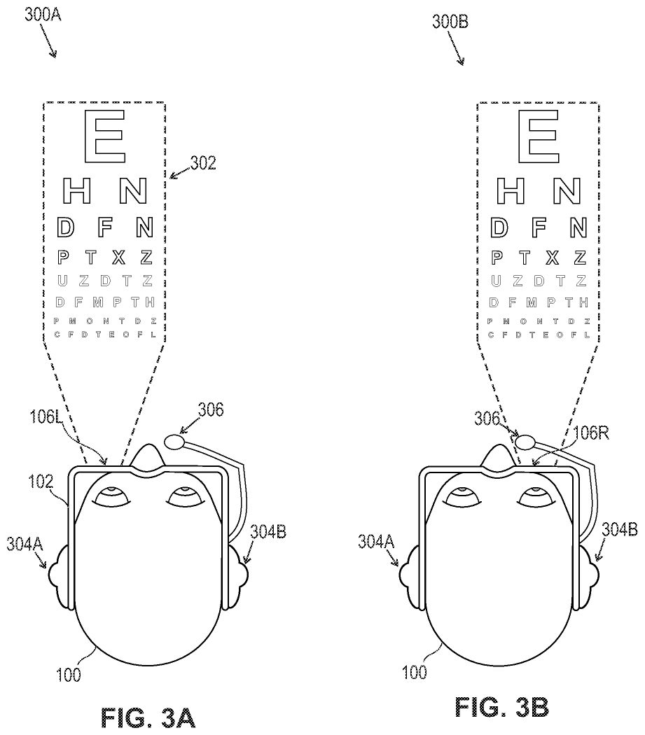

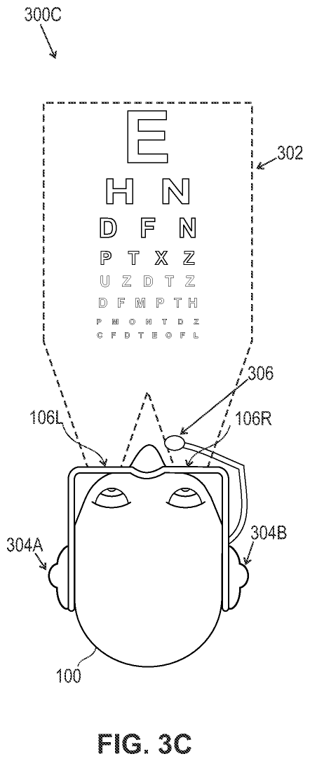

FIGS. 3A, 3B and 3C are aerial view representations 300A, 300B and 300C of a patient 100 utilizing a HMD to take an eye examination in accordance with the disclosure. In the example shown in FIGS. 3A, 3B and 3C, a visual acuity eye test is administered which is used to determine the smallest letters a patient can read on a standardized chart or a card that is held 20 feet (about 6 meters) away. In general, visual acuity is measured by showing five symbols of a selected optotype in a line which is rendered as a virtual eye chart inside a virtual room to a patient. Thus, FIG. 3A is an aerial view 300A of visual acuity testing of the left eye of the patient 100, whereas FIG. 3B is an aerial view 300B of visual acuity testing of the right eye of the patient, and FIG. 3C is an aerial view 300C of visual acuity testing of both the left and right eyes of the patient 100, as may be experienced in some embodiments. In this example, the HMD 102 is configured to project an image of a Snellen chart 302 to each eye separately and then to both eyes together.

In accordance with methods described herein, the patient 100 wears the HMD 102 so that optical display surfaces 106R and 106L are positioned in front of his or her right eye and left eye, respectively, and so that speakers 304A and 304B are positioned over his or her ears. In addition, the patient adjusts the microphone 306 to be positioned in front of his or her mouth. A visual acuity test can then begin with the patient first listening to prerecorded audio instructions explaining the eye testing process and/or procedures that will be used during the eye examination.

A visual acuity module for conducting this eye test may be downloaded from an application store (an App store, such as iTunes™ or Google Play™) to the HMD 102 and then utilized to test the patient's eyes. In some implementations, an eye-tracking feature of the HMD 102 and/or of the visual acuity module is used to ensure compliance by the patient 100 with the testing procedures. Specifically, as instructions are provided to the patient concerning reading a particular line of the virtual eye chart 302, which is displayed on an optical display surface (106R, 106L or both), an integrated camera (not shown) of the HMD 102 tracks infra-red (IR) reflections from the patient's eye and processes that data to determine where the patient's eye is looking at any point in time during the eye test.

In some implementations, the HMD 102 may display symbols of a virtual eye chart, such as the virtual Snellen chart 302, on the optical display surfaces 106R, 106L that may be arranged randomly to restrict memorization, and the eye-tracking feature may be utilized to detect squinting of the patient's eyes. In addition, during the visual acuity eye test, the patient may be prompted to provide audio responses which can be received by the microphone 306 and stored as an audio data file, and/or to provide physical responses by using a hand controller (not shown) that can generate motion data which also may be stored.

In some embodiments, a physician in charge of a patient may assign a particular optotype, which consists of figures or letters of different or variable sizes, for use to test the visual acuity of the patient. In other implementations, the patient may choose between various optotypes. Optotypes that may be selected to test visual acuity include a Landolt C optotype (used frequently to test high-contrast visual acuity), a Tumbling E optotype (which may be used to test children or illiterate patients), a Snellen chart, or a standard ETDRS chart (or Log MAR chart) consisting of rows of letters typically used by optometrist to test visual acuity. In each case as shown in FIGS. 3A, 3B and 3C, the shape, size and spacing of the letters (or symbols) are compliant with eye test standards.

In FIGS. 3A and 3B, the visual acuity module projects the virtual Snellen chart 302 to each eye of the patient separately, which simulates an ophthalmologist or optometrist covering one eye of the patient with a paddle or other object (while testing the vision of the other eye). Consequently, in this implementation the HMD 102 may use an electronics module (not shown) to control what can be seen by each of the right and left eyes while eye tests are being performed. Some tests may also require the HMD 102 to present an image 208 to both eyes simultaneously, as shown in FIG. 3C.

Some alternatives to traditional eye examinations typically fail to adequately control or account for conditions that impact test results, such as glare, image brightness, humidity, and the like. For example, some Smartphone applications are unable to account for glare on the screen or to determine whether or not the patient has completely covered one eye during testing of the other eye. However, testing utilizing an HMD as described herein allows for conditions and contaminants to be closely monitored and/or kept consistent. The optical display surfaces of the HMD 102 depicted in FIGS. 3A-3C, for example, may be configured for preventing glare while conducting the eye examination.

In addition, if a patient uses corrective lenses, then that patient may choose to wear their glasses underneath the HMD during testing. Alternately, in some implementations that patient's lens specification(s) can be utilized by the HMD to augment the virtual environment and virtual displays in the same manner that the patient's corrective lenses would be serve. In addition, in some embodiments the patient takes the visual acuity test by following the instructions supplied by an audio recording, wherein the visual acuity of the right eye is measured first, then the visual acuity of the left eye is measured, and finally both eyes are tested together for visual acuity.

In disclosed embodiments, the patient does not have to look away from the virtual eye chart 302 at any time during the eye examination, and any verbal or physical responses can be recorded and/or analyzed to determine whether the patient correctly identified the symbols and/or letters of a particular optotype. In particular, the size of the rendered virtual symbols follows a monotonically decreasing scheme, and if the patient succeeds in reading more than half of the symbols in a particular line then the size of the new line or next line is decreased. However, a failure by the patient to successfully read more than half the virtual symbols in a particular line results in a new virtual line to be displayed by the HMD 102 that is sized to be midway between the current virtual symbol line and the last successfully read virtual symbol line, which process is continued until the size difference becomes too small for the patient to read.

With regard to the visual acuity test, in some embodiments if there is a significant difference between the performance of consecutive tests, the HMD 102 transmits a warning message to the physician's electronic device that includes the results. In addition, in some implementations all of the lines displayed to the patient, the visual acuity in log MAR, and all audio responses of the patient, may be stored for future comparison and/or analysis. In addition, if a patient is unable to see even the biggest or largest virtual eye test line displayed by the HMD 102, then in some implementations a low vision acuity test is conducted, the results saved and transmitted to the physician for analysis and treatment.

In accordance with a thorough eye examination, a low-vision acuity test may be administered to a patient. Thus, a low-visional acuity test module may be downloaded to the HMD 102 which, in some embodiments during testing, displays a hand in a virtual environment to the patient (not shown). The hand movement may be regulated between some levels (for example, hand waving) and the patient is asked to perceive the levels and provide an audio or other response. Next, a penlight may be shined on the patient's eyes in a monocular manner to detect whether the eye can perceive the orientation of the light. The results of these eye tests are recorded. If the patient is unable to provide responses to the hand movements and/or to the orientation of the penlight during testing, then the low-vision acuity test may display a random number of virtual fingers, and the patient prompted to audibly provide the number of fingers raised. The patient's response is recorded, and if his or her performance is significantly different, the physician is notified.

In some implementations, a near visual acuity module may be downloaded to the HMD 102 to gauge a patient's near working distance and illumination comfort. In some embodiments, a patient has significant control over this test as the patient is first instructed to adjust the light of the virtual room to his or her comfort level. After adjustment, a virtual card is attached to a hand-held motion controller (not shown in the drawings) and the patient has the freedom to bring the card close for improved visibility. The patient reads a virtual paragraph displayed on the virtual card appearing on the optical display surfaces 106R, 106L out loud, and the recording of that reading is then analyzed to determine visual acuity of the patient. In some embodiments, the distance of the virtual card from patient's eyes, the visual acuity in log MAR, the time taken to complete testing, and the recording and displayed texts are all stored for future analysis.

Another eye test module is a contrast sensitivity module which can be downloaded to the HMD 102. Contrast sensitivity testing measures how well the patient's eyes can distinguish between finer and finer light increments compared to a dark (contrast) pattern. Such eye testing is not typically administered as part of a routine eye exam, but an eye doctor may recommend it, or a patient may request it if the patient has a specific visual complaint concerning visual contrast. In some embodiments, the contrast sensitivity module tests a patient's eyes by displaying a grey noise representation having a standardized grating (for example, a frame of parallel bars) and contrast on a completely white background. The noise is then smoothly moved across the display screen and the patient must track the noise through the random direction, grating frequency and contrast changes. This is verified by tracking the patient's eye gaze direction, position, velocity and acceleration and comparing it with the actual motion of the noise. The continuous success of the patient in tracking the actual motion of the noise results in the test continuing by decreasing the contrast and spatial frequency. However, after significant deviation between the two motions, the contrast is increased. The monocular test produces two contrast sensitivity functions for the two eyes of the patient.

Another eye test module is a color vision module which can be downloaded to the HMD 102 and administered to the patient 100. The color vision test, which is known as the Ishihara color test, measures a patient's ability to tell the difference among colors. In some embodiments, each of the patient's eyes is tested separately by being shown a series of virtual test cards that each contains a multicolored dot pattern. A number or symbol appears within each color pattern which the patient is asked to identify. The numbers, shapes, and symbols are easy to distinguish from the surrounding dot patterns if the patient has normal color vision. If there is a color vision impairment, then the patient might not be able to see the numbers and/or symbols. The color visions module installed on the HMD 102 may instruct the patient to audibly provide answers concerning perceived numbers and symbols which may be stored and may also ask the patient to describe a particular color's intensity as perceived by one eye versus the other eye. The color vision test may reveal that the patient has a normal color vision but still experiences a loss of color intensity in one eye or the other. If the patient does not pass this test, he or she may have poor color vision and/or may be color blind.

An example of yet another eye test that may be administered to the patient via the HMD 102 is a visual field test. A visual field test module may be downloaded to the HMD and when launched prompts the patient to look at a specified fixation point in a virtual environment. If the patient's gaze deviates from the fixation point, then no further stimulus is presented until the gaze settles down. The visual field stimuli are then reoriented and the patient is presented with a new fixation point. The patient's eye response to stimuli in a thirty-degree (30°) central visual field is objective as the patient's eye response data may be recorded with an electroencephalogram (EEG). In some implementations, the patient wears a cap with an array of EEG electrodes (not shown in the drawings), a series of visual and auditory stimuli are presented to the patient, and then EEG patterns are recorded as baseline. Next, the patient is presented with the Visual Field assessment and stimuli are presented while EEG recordings identified as responses to the stimulus are collected. At the presentation of the stimulus, if the EEG recording shows a response, the stimulus will be considered as seen by the patient. If the signal in the EEG recording at the time of the presentation of the stimulus is nonexistent, then the stimulus is considered as missed by the patient.

In addition, in some embodiments two protocols are followed to measure the sensitivity of the rod and cone photoreceptors of each eye of the patient separately. In the cone specific protocol, the patient's eyes are adapted to a dim blue background for five minutes and then red stimuli are presented against this background. In the rod specific protocol, a period of twenty minutes is required to adapt the patient's eyes to a dark background after which a dim blue stimuli are presented (against the dim blue background). A sensitivity map of the cone and rod receptors of the patient's eyes is then overlaid on the individual's retinal scan to determine amplitude and/or statistics of the responses of the rods or cones at various regions of the retina, resulting in a visual fields map. The results data of the patient's visual field test are then stored for later analysis.

Another eye test module is an Amsler grid test module which can be downloaded to the HMD 102 and then administered to the patient. An Amsler grid is used to check whether lines look wavy or distorted, or whether areas of the visual field are missing. An eye doctor will use such a test to identify the presence of scotomas in a patient's visual field, which indicates changes in the macula. At the start of the test, a virtual Amsler grid may be displayed to both eyes of the patient, and later may be administered to one eye at a time (and the patient may wear his or her corrective lenses during testing if normally worn). Thus, in an implementation, the HMD 102 displays a virtual Amsler grid about 28 centimeters (cm) to 30 cm away on the optical display surfaces 106R, 106L, and the patient is asked to look at a dot (fixation point) in the center of the grid. While looking at the fixation point, if the straight grid lines appear to be distorted in one of the eyes, the patient reports or selects which eye is seeing a distortion (has a problem), and the eyes are tracked for fixation loss.

In some embodiments, the Amsler grid test displays a separate but identical grid to the patient's healthy eye and then the patient is asked to emulate the metamorphopsia (distorted vision) of the deficient eye on the healthy eye using one or more motion controllers. This grid manipulation is constructed as a Gaussian mixture model of distortion, and when the two grids look similar, the patient has completed the test and the Gaussian mixture model parameters are stored. In some implementations, if the parameters vary significantly compared to a previous patient eye test, then the physician is notified.

In some embodiments, the Amsler grid test also includes the HMD 102 applying an inverse distortion onto the optical display surface in front of the deficient eye of the patient. The Amsler grid module includes instructions that causes the HMD 102 to ask the patient if the application of the inverse distortion corrects the distortion, but if this is not effective then a virtual scotoma is presented to the region of the eye to suppress the metamorphopsia and let the other eye take over in the affected area. In implementations, gaze tracking ensures that the position of the scotoma and the inverse filter is always stationary with respect to the wandering eyes of the patient.

It should be noted that, in implementations discussed herein concerning storing of a patient's eye test data and/or other data and/or processor-executable instructions, storage devices such as a local storage device (which may be a component of the HMD) and/or an edge computing device and/or a remote storage device may be utilized. Accordingly, in some implementations a patient's eye test data may be transmitted to an edge computing device for storing and/or transmitted to a remote server computer for storage in a remote storage device (such as a patient database) and/or transmitted to a cloud storage system that may include one or more cloud storage device(s) (not shown, which may include on or more patient database(s)). When the results data of the patient's various eye tests are stored locally in a storage device of the HMD device for later analysis and/or processing, at some point in time after the eye tests are concluded the HMD may be configured to transmit such eye test results to, for example, a computer device of an eye doctor or other eye professional for review. In some embodiments, the patient's eye test data may be transmitted before and/or after processing to a remote server computer or to a cloud storage system for storage in one or more patient database(s).

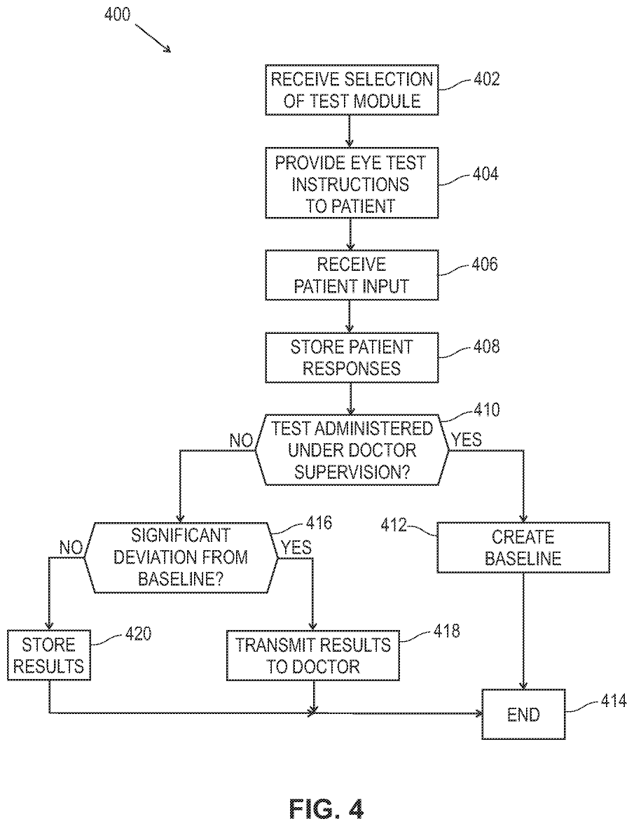

FIG. 4 is a flowchart of an eye testing process 400 in accordance with this disclosure. In an implementation, the HMD receives 402 selection of a test module and then provides 404 eye test instructions to the patient who is wearing the HMD. The instructions may be delivered audibly if the HMD is outfitted with speakers or headphones or may be displayed to the patient. Next, the HMD receives patient responses or input 406, stores 408 those responses and next determines 410 whether the eye test was administered by an eye doctor. If so, then a baseline is created 412 which is stored in a storage device for analysis by the eye doctor and for use in future when the patient again undergoes eye testing, and the process ends 414. As mentioned earlier, the eye test data may be stored in a local storage device and/or transmitted to a remote computer device for analysis by an eye doctor or other professional.

Referring again to step 410, if the eye test was not administered by an eye doctor, then the HMD determines 416 is there is a significant deviation from a past baseline measurement. If so, then the eye test results are transmitted 418 to of an eye doctor of the patient and the process ends. For example, the eye test results may be transmitted 418 to a laptop computer of the patient's eye doctor via the Internet or other computer network. However, if there was no significant deviation from a baseline measurement, then the “unchanged” eye test results are stored 420 and the process ends 414. In some embodiments, the unchanged eye test results are also transmitted to of an eye doctor of the patient. In some implementations, when data is transmitted to the eye doctor it may include a patient identifier (patient ID), a date of the testing, data corresponding to the eye test elements displayed to the patient during the eye tests, data corresponding to the user responses, and a time the patient took to read various portions or one or more eye tests.

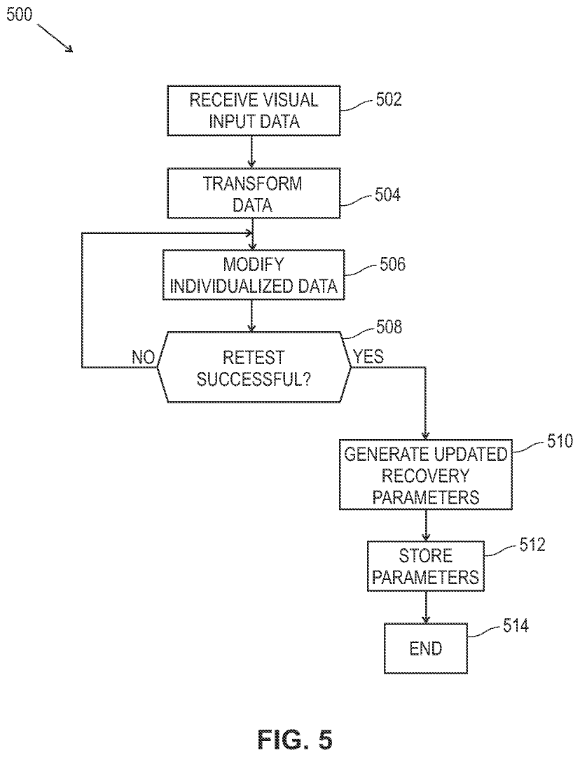

FIG. 5 is a flowchart of a process for generating recovery parameters 500 for a patient based on one or more eye examinations in accordance with disclosed processes. Thus, the HMD receives 502 visual input data from a patient undergoing an eye test, and transforms 504 the visual input data into recovery parameters based on patient vision parameters. In some embodiments, the transformation includes generating recovery parameters that redistribute the color palate of the scene to accommodate patients who suffer from color sensitivity losses, for example, color blindness or cataracts. The transformation may also include spatial transformations, such as rotation and/or displacement, of pixels in the patient's affected regions of the visual field(s). For example, in the case of advanced glaucoma wherein the patient suffers from loss of peripheral visual field (e.g. “tunnel vision”), the pixels at the periphery of the scene may be moved to more central locations while preserving the central field as undisturbed as possible.

Referring again to FIG. 5, individual vision parameters are next modified 506 or tweaked based on patient interaction with the HMD and the eye test module, and then the HMD determines 508 if a retest of the patient is successful. If so, then the HMD generates 510 updated recovery parameters and stores 512 the updated recovery parameters. The process then ends 514. A successful retest is one in which the patient can present sufficient visual function after the parameters of their visual loss have been utilized to perform appropriate transformations on the visual input for the patient to operate close to normal. If the patient cannot present sufficient visual function in the retest phase, the retest is considered failed and a new set of parameters is determined by the patient. In order to provide new parameters, the patient will perform the original tests (for example, visual acuity, visual field, and the like tests) assigned to them. Referring again to FIG. 5, if in step 508 the retest of the patient is not successful (unsuccessful) then the process branches back to step 506 wherein the individualized patient parameters are again modified.

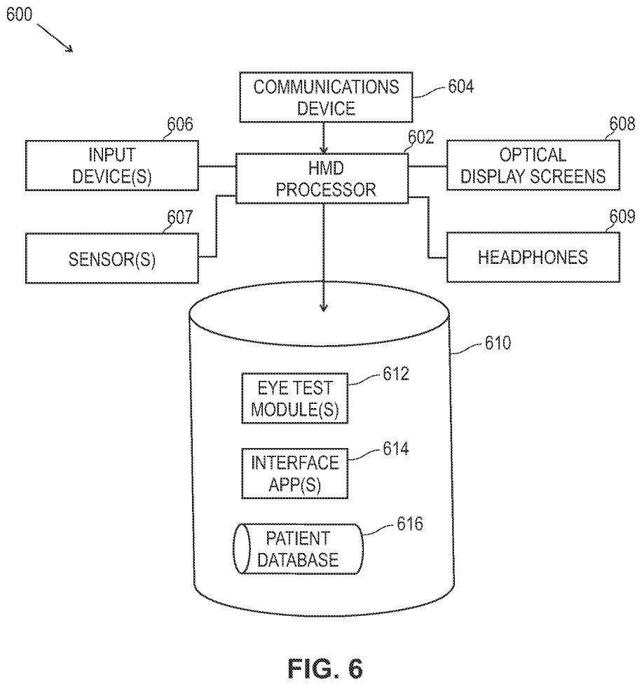

FIG. 6 is a block diagram of an example embodiment of the components of an HMD 600 of a type configured to operate in a manner consistent with processes described herein. The HMD 600 includes a HMD processor 602 operatively coupled to a communication device 604, one or more input devices 606, one or more sensors 607, optical display surfaces (one for each eye of a patient) 608, headphones 609 (or speakers; one for each ear of the patient), and a storage device 610. The HMD processor 602 may constitute one or more processors, which may be custom designed and/or optimized to execute HMD instructions and/or processor-executable steps, which may be contained in program instructions so as to control the HMD 600 provide desired functionality.

Communication device 604 may be used to facilitate communication with, for example, other electronic or digital devices such as other components of the system 200 shown in FIG. 2. Thus, communication device 604 may comprise various and/or numerous communication ports (not separately shown), to allow the HMD 600 to communicate simultaneously with a considerable number of other computers or electronic devices, and/or to simultaneously handle numerous functions including eye testing functions. The communication device 604 may also be configured for wireless communications and/or wired communications via various different types of private networks and/or public networks, such as the Internet.

The input devices 606 may include one or more of any type of peripheral device typically used to input data into an HMD or to a computer. For example, the input device 606 may include a microphone and/or hand controller(s), and/or a touchscreen. The one or more sensors 607 may include, for example, a camera to record patient interactions with a testing environment during eye testing, and/or a temperature sensor to record the testing environment temperature.

Storage device 610 may be any appropriate information storage device, including combinations of magnetic storage devices (e.g., hard disk drives), optical storage devices such as CDs and/or DVDs, and/or semiconductor memory devices such as Random Access Memory (RAM) devices and Read Only Memory (ROM) devices, solid state drives (SSDs), as well as flash memory or other type of memory or storage device. Any one or more of such information storage devices may be considered to be a non-transitory computer-readable storage medium or computer usable medium or memory.

Storage device 610 stores one or more programs, program modules and/or applications (Apps) for controlling the HMD processor 602. The programs, program modules and/or Apps comprise program instructions (which may be referred to as computer readable program code means) that contain processor-executable process steps of the HMD 600 which are executed by the HMD processor 602 to cause the HMD 600 to function as described herein.

The programs may include one or more conventional operating systems (not shown) that control the HMD processor 602 so as to manage and coordinate activities and sharing of resources in the HMD 600, and to serve as a host for application programs (such as those described below) that run on the HMD 600.

The storage device 610 may also store one or more eye test modules 612 which include processor-executable instructions for administering one or more eye tests as described herein to a patient, recording the outcome(s), and in some cases contacting an eye doctor and/or transmitting information and/or eye test data to, for example, a remote computer or computer network. In addition, the storage device 610 may also store interface applications 614 which include processor executable instructions for providing software interfaces (such as graphical user interface(s)) to facilitate interaction(s) between a patient being tested by use of one or more eye test modules and other components of the system 200.

The storage device 610 may also store, and HMD 600 may also execute, other programs, which are not shown. For example, such other programs may include HMD display device drivers, database management software, and the like.

Moreover, the storage device 610 may also store a patient data database 616 for storing patient identification data, patient eye test data such as results of specific eye tests, whether or not an eye doctor was involved with testing and/or notified of the eye test results, and the like. In addition, one or more further databases (not shown) which may be needed for operation of the HMD 600 may also be included.

In disclosed embodiments, the eye test modules which include eye tests administered via an HMD described herein advantageously conform to well-established visual acuity measurement protocols that include illumination, symbol size, symbol spacing and symbol contrast. In addition, eye test methods disclosed herein receive patient input via audio responses and/or motion controller button(s), and in some implementations the audio input may be compared to input provided by a machine learning protocol. Eye test results data may be compared with previous testing results and/or to an adjusted baseline and any significant change in performance may be noted, and/or stored, and/or transmitted to an eye professional such as an eye doctor for analysis and the like. In addition, a low vision test module is beneficially available to determine the stage of deterioration of a patient's perception. Also, in some implementations, difficulty in reading (due to confusion, and/or taking longer than normal) is considered when analyzing verbal responses from a patient, and individual reading patterns are also considered and stored to discard bias. Moreover, in implementations a patient has the freedom to adjust certain variables during testing, such as illumination, to assist reading.

In some embodiments with regard to some eye tests, as described above contrast sensitivity is measured by moving a noise across the user's field of vision, gaze tracking determines whether the user was able to follow the motion, and smooth tracking is used which makes the testing shorter, objective and easier. Also, an assessment of a patient's perceptual deficit is accomplished by having the patient interact with an Amsler grid, and eye deficit parameters are used for vision recovery purposes. Moreover, in implementations, separate perimetry testing is used for the cone and rod photoreceptors, for analysis purposes a sensitivity map is overlaid on an existing retinal scan, and any fixation loss is noted and/or stored by using gaze tracking and adjusting stimulus and fixation positions.

As used herein, the term “computer” should be understood to encompass a single computer or two or more computers in communication with each other.

As used herein, the term “processor” should be understood to encompass a single processor or two or more processors in communication with each other.

As used herein, the term “memory” should be understood to encompass a single memory or storage device or two or more memories or storage devices.

As used herein, a “server” includes a computer device or system that responds to numerous requests for service from other devices.

The above descriptions and illustrations of processes herein should not be considered to imply a fixed order for performing the process steps. Rather, the process steps may be performed in any order that is practicable, including simultaneous performance of at least some steps and/or omission of steps.

Although the present disclosure has been described in connection with specific example embodiments, it should be understood that various changes, substitutions, and alterations apparent to those skilled in the art can be made to the disclosed embodiments without departing from the spirit and scope of the disclosure.

What is claimed is:

1. A method for administering modular eye tests to a patient via a head mounted display (HMD) device comprising:
receiving, by a head-mounted display (HMD) device via one of an input device and a communications device, selection of a test module associated with a specific eye test which has been downloaded from an application store to the HMD device;
providing, by the HMD device to an output device, instructions to a patient concerning the specific eye test;
receiving, by the HMD device via the input device, patient input data responsive to the specific eye test instructions; and
storing, by the HMD device, the patient input data in a patient database.

2. The method of claim 1, further comprising:
determining, by the HMD device, that the test module was selected by an eye doctor; and
generating, by the HMD device, a baseline model of the specific eye test results of the patient.

3. The method of claim 2, further comprising storing, by the HMD device, the baseline model of the patient in the patient database.

4. The method of claim 1, further comprising:
determining, by the HMD device, that the test module was selected by the patient;
determining, by the HMD device, that the specific eye test results significantly deviate from a baseline model of the patient; and
transmitting, by the HMD device via a communications device, the specific eye test results to an eye doctor associated with the patient.

5. The method of claim 1, further comprising:
determining, by the HMD device, that the test module was selected by the patient;
determining, by the HMD device, that the specific eye test results do not significantly deviate from a baseline model of the patient; and
storing, by the HMD device, the specific eye test results in the patient database.

6. The method of claim 1, further comprising:
transforming, by the HMD device based on patient vision parameters, the patient input data into recovery parameters;
modifying, by the HMD device, individual patient vision parameters based on further patient input data;
retesting, by the HMD device, the patient;
determining, by the HMD device, that the retesting of the patient is successful;
generating, by the HMD device, updated recovery parameters; and
storing, by the HMD device in the patient database, the updated recovery parameters.

7. The method of claim 1, wherein storing the patient input data in a patient database comprises one of storing, by the HMD device, the patient input data in a local storage device or transmitting, by the HMD device, the patient input data to at least one of an edge computing device and a remote storage device.

8. A method for administering modular eye tests to a patient via a head mounted display (HMD) device comprising:
receiving, by a head-mounted display (HMD) device from an application store via a communications device, a test module associated with a specific eye test;
providing, by the HMD device via a speaker to a patient, instructions concerning the specific eye test;
receiving, by a microphone of the HMD device from the patient, audio response data responsive to the specific eye test instructions; and
storing, by the HMD device, the audio response data in a patient database.

9. The method of claim 8, further comprising:
determining, by the HMD device, that the test module was selected by an eye doctor; and
generating, by the HMD device, a baseline model of the specific eye test results of the patient.

10. The method of claim 8, wherein storing the audio response data in a patient database comprises one of storing, by the HMD device, the audio response data in a local storage device or transmitting, by the HMD device, the audio response data to at least one of an edge computing device and a remote storage device.

11. An eye examination system for administering modular eye tests to a patient comprising:
a head mounted display (HMD) device comprising an HMD processor, an input device operably connected to the HMD processor, a communications device operably connected to the HMD processor, optical display screens operably connected to the HMD processor, at least one speaker operably connected to the HMD processor, and a storage device operably connected to the HMD processor, wherein the HMD device is configured for wearing by a patient;
a computer system operably connected to the HMD device via a communications network; and
at least one electronic device operably connected to the computer system;
wherein the storage device comprises processor executable instructions which when executed causes the HMD processor to:
receive, via one of the input device and the communications device, selection of a test module associated with a specific eye test which has been downloaded from an application store to the HMD device;
provide instructions to the patient via one of the optical display screens or the at least one speaker concerning the specific eye test;
receive, via the input device, patient input data responsive to the specific eye test instructions; and
store the patient input data in the storage device.

12. The eye examination system of claim 11, wherein the storage device comprises further processor executable instructions which when executed causes the HMD processor to:
determine that the test module was selected by an eye doctor; and
generate a baseline model of the specific eye test results of the patient; and
store the baseline model of the patient in the storage device.

13. The eye examination system of claim 11, further comprising a communications device operably connected to the HMD processor, and wherein the storage device comprises further processor executable instructions which when executed causes the HMD processor to:
determine that the test module was selected by the patient;
determine that the specific eye test results significantly deviate from a baseline model of the patient; and
transmit the specific eye test results to an eye doctor associated with the patient.

14. The eye examination system of claim 11, wherein the storage device comprises further processor executable instructions which when executed causes the HMD processor to:
determine that the test module was selected by the patient;
determine that the specific eye test results do not significantly deviate from a baseline model of the patient; and
store the specific eye test results in the storage device.

15. The eye examination system of claim 11, wherein the storage device comprises further processor executable instructions which when executed causes the HMD processor to:
transform, based on patient vision parameters, the patient input data into recovery parameters;
modify individual patient vision parameters based on further patient input data received via the input device;
retest the patient;
determine that the retesting of the patient is successful;
generate updated recovery parameters; and
store the updated recovery parameters in the storage device.

16. The eye examination system of claim 11, further comprising at least one of a remote storage device and an edge storage device, and wherein the storage device comprises further processor executable instructions which when executed causes the HMD processor to transmit the patient input data to at least one of an edge computing device and a remote storage device.

17. The eye examination system of claim 11, wherein the input device of the HMD device comprises at least one of an optical sensor, a temperature sensor, a humidity sensor, and a light sensor.

18. An eye examination system for administering modular eye tests to a patient comprising:
a head mounted display (HMD) device comprising an HMD processor, an input device operably connected to the HMD processor, optical display screens operably connected to the HMD processor, a speaker operably connected to the HMD processor, a communications device operably connected to the HMD processor and a storage device operably connected to the HMD processor, wherein the HMD device is configured for wearing by a patient;
a computer system operably connected to the HMD device via a communications network; and
at least one electronic device operably connected to the computer system;
wherein the storage device comprises processor executable instructions which when executed causes the HMD processor to:
download, via the communications device, a test module associated with a specific eye test from an application store;
provide, via the speaker, audible instructions concerning the specific eye test to a patient;
receive, via the input device from the patient, audio response data responsive to the specific eye test instructions; and
store the audio response data in the storage device.

19. The eye examination system of claim 18, wherein the storage device comprises further processor executable instructions which when executed causes the HMD processor to:
determine that the downloaded test module was selected by an eye doctor; and
generate a baseline model of the specific eye test results of the patient.

20. The eye examination system of claim 18, wherein the instructions for storing the audio response data in a patient database comprises processor executable instructions which when executed causes the HMD processor to store the audio response data in a local storage device or transmit the audio response data to at least one of an edge computing device and a remote storage device.

21. The eye examination system of claim 18, wherein the input device of the HMD device comprises at least one of an optical sensor, a temperature sensor, a humidity sensor, and a light sensor.
